SEQUENCE LISTING (8 sequences)

SEQ ID NO: 1 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 2 - protein, 355 aa, Homo sapiens
MDFLHRSGVLIIHHLQEDYRTYYGFLNFMSNVGDPRNIFSIYFPLWFQLNQNVGTKMIWVAVIGDWFNLIFKWILFGHRPYWWIQETEIYPNHSSPCLEQFPTTCETGPGSPSGHAMGSSCVWYVMVTAALSYTISRMEESSVTLHRLTWSFLWSVFWLIQISVCISRVFIATHFPHQVILGVIGGMLVAEAFEHTPGVHMASLSVYLKTNVFLFLFALGFYLLLRLFGIDLLWSVPIAKKWCANPDWIHIDSTPFAGLVRNLGVLFGLGFAINSEMFLRSCQGENGTKPSFRLLCALTSLTTMQLYRFIKIPTHAEPLFYLLSFCKSASIPLMVVALIPYCVHMLMRPGDKKTK

SEQ ID NO: 3 - protein, 24 aa, Homo sapiens
MDFLHRNGVLIIQHLQKDYRAYYT

SEQ ID NO: 4 - protein, 20 aa, Homo sapiens
MLVAEAFEHTPGIQTASLGT

SEQ ID NO: 5 - protein, 10 aa, Homo sapiens
LLSCRGGNNY

SEQ ID NO: 6 - protein, 12 aa, Homo sapiens
HMLMKQSGKKSQ

SEQ ID NO: 7 - protein, 102 aa, Homo sapiens
MDFLHRNGVLIIQHLQKDYRAYYTFLNFMSNVGDPRNIFFIYFPLCFQFNQTVGTKMIWVAVIGDWLNLIFKWKSIWPCNGRILCLVCHGNRCPEPHCLWDG

SEQ ID NO: 8 - protein, 14 aa, Homo sapiens
MLVAEAFEHTPGIQ

(19) United States
(12) Patent Application Publication
(10) Pub. No.: US 2011/0286977 A1
(43) Pub. Date: Nov. 24, 2011

(54) G6PC2-Encoded Beta Cell Surface Tags

(75) Inventors: Bart Roep, Leiden (NL); Jacob Hald, Birkerod (DK); Ole Dragsbaek Madsen, Soborg (DK)

(73) Assignee: Novo nordisk A/S, Begasvaerd (DK)

(21) Appl. No.: 13/119,030

(22) PCT Filed: Sep. 30, 2009
(86) PCT No.: PCT/EP2009/062696

§ 371 Date: Jul. 21, 2011

Related U.S. Application Data
(60) Provisional application No. 61/103,668, filed on Oct. 8, 2008.

(30) Foreign Application Priority Data
Sep. 30, 2008 (EP) 08165500.3

(51) Int. Cl. A61K 35/39; A61P 3/10; C12N 9/16; C07K 16/40; G01N 33/573; C12N 5/071

(52) U.S. Cl. 424/93.7; 435/7.21; 435/325; 435/196; 530/387.9

(57) ABSTRACT

The invention relates to G6PC2-encoded beta cell surface markers, methods of identifying and obtaining a culture cells comprising fully differentiated beta cells. Also contemplated is a method of sorting such cells, isolated cells and compositions thereof.

### FIELD OF THE INVENTION

The invention relates to selective cell surface markers, which are extracellular parts of islet-specific Glucose-6-phosphate catalytic-subunit Related Protein (IGRP) encoded by G6PC2, and which permit the selection and quantification of a unique subset of cells that comprise the fully differentiated pancreatic endocrine beta cell phenotype. In one embodiment the G6PC2-encoded beta cell surface tags are splice variants. The sequences hereof, compositions, cell cultures, and cell populations comprising fully differentiated beta cells are also contemplated as well as methods of producing fully differentiated beta cells and detecting fully differentiated beta cells.

### BACKGROUND OF THE INVENTION

Beta cell transplantation holds great promise to improve treatment of Type 1 diabetes but a number of obstacles need to be overcome first. Among these is the scarcity of available donor islets. Embryonic stem (ES) cell derived beta cells can in principle supply unlimited numbers of beta cells for transplantation but reliable protocols for generating fully functional beta cells are not yet developed. Formation of definitive endoderm (DE) cells from embryonic stem cells has been reported for both mouse and human ES cells in, e.g., WO 2005/116073, WO 2005/063971, and US 2006/0148081. Efficient generation of pancreatic endoderm (PE) cells from e.g. DE cells is advantageous for generation of insulin-producing beta cells for the treatment of diabetes.

In attempting to cultivate fully differentiated pancreatic islet cells, the objective has long been to isolate pancreatic cells including pancreatic endocrine pre-progenitor cells that are capable of differentiating into pancreatic beta cells or islets. One important step in isolation of the pancreatic endocrine lineage from the exocrine lineage would be to identify recognizable cell markers, specific for the pancreatic endocrine pre-progenitor cells and/or progeny thereof—and in particular markers that are specific for the mature insulin producing beta cell. Both intracellular and extracellular markers have been investigated for this purpose. Intracellular markers, particularly transcription factors detected in embryonic pancreatic cells that develop into fully differentiated islet cells, have been extensively studied as progenitor markers. Transcription factors, such as Pdx-1, Ngn3, Pax6, and Isl-1, for example, have been studied. They are expressed in cells that are programmed during embryonic development to become pancreatic endocrine cells. However, these intracellular markers offer less practical value than extracellular markers, because analysis of expression of those markers requires either the killing of the cells or permanent modification of the cells by genetic engineering of reporter genes into the cells.

Once identified, extracellular markers would offer the advantage that the cells expressing the marker can be sorted under sterile conditions and kept alive. Epithelial cell adhesion molecules, such as Ep-CAM and integrins have been investigated as pancreatic islet progenitor markers.

### DESCRIPTION OF THE DRAWINGS

FIG. 1 depicts the overall organization of the full length human G6PC2-encoded peptide—indicating the transmembrane stretches as well as the 5 ER-luminal domains and the 5 cytoplasmic domains. Exon-exon boundaries are marked by grey circles and arrows. Amino acid residues predicted to comprise the active centre are denoted by filled-in black circles. The Asn-linked glycosylation site at residues 92-94 acts as an acceptor for oligosaccharides.

FIG. 2 shows full length ClustalW alignment of mouse and human G6PC2-encoded peptide. In the line between the human and the mouse protein sequence identical residues are indicated with a one-letter code for the amino acid, conservative substitutions are indicated with “+” and non-conservative substitutions are indicated with “ ”, i.e. a blank space.

### SUMMARY OF THE INVENTION

In one embodiment the invention relates to an isolated peptide encoded by G6PC2, wherein said peptide comprises i) an extracellular part and ii) a deletion in one or more exons of G6PC2, provided that said peptide is not MDFLHRNGVLIIQHLQKDYRAYYTFLNFMSNVGDPRNIFFIYFPLCFQFNQTVGTKMIWVAVIGDWLNLIFKWKSIWPCNGRILCLVCHGNRCPEPHCLWDG. In one embodiment the invention relates to an isolated peptide encoded by G6PC2, wherein said peptide comprises i) an extracellular part and ii) a deletion in one or more exons of G6PC2 as well as an isolated nucleotide encoding said peptide and an isolated cell expressing said peptide. In one embodiment the invention relates to a method of identification of fully differentiated beta cells, the method comprising contacting a cell population comprising pancreatic cells with a reagent binding a G6PC2-encoded beta cell surface tag.

In one embodiment the invention relates to a method of obtaining a culture of fully differentiated beta cells, the method comprising: contacting a cell population comprising pancreatic cells with a reagent binding a G6PC2-encoded beta cell surface tag and separating the cells that binds the reagent binding a G6PC2-encoded beta cell surface tag in a fraction of cells positive for a G6PC2-encoded beta cell surface tag from cells that do not bind the reagent binding a G6PC2-encoded beta cell surface tag.

In one embodiment the invention relates to a method of providing pancreatic endocrine function to a mammal deficient in its production of at least one pancreatic hormone wherein cells are obtained by the method as defined herein, the method further comprising the steps of: implanting into the mammal the obtained cells in an amount sufficient to produce a measurable amount of at least one pancreatic hormone in the mammal. In one embodiment said at least one pancreatic hormone is insulin. In one embodiment the mammal is a human being

In one embodiment the invention relates to a method of quantifying cells positive for a G6PC2-encoded beta cell surface tag comprising pancreatic cells by a) contacting the cells with a reagent binding a G6PC2-encoded beta cell surface tag; and b) determining the quantity of cells that exhibit a G6PC2-encoded beta cell surface tag as a cell surface marker (cells positive for G6PC2-encoded beta cell surface tag).

In one embodiment the invention relates to a method for the optimisation of an in vitro protocol, wherein the number of cells expressing a G6PC2-encoded beta cell surface tag (cells positive for a G6PC2-encoded beta cell surface tag) is periodically monitored.

In one embodiment the invention relates to an isolated fully differentiated beta cell obtained by a method as defined herein.

In one embodiment the invention relates to a composition comprising isolated fully differentiated beta cells obtained by a method as defined herein.

In one embodiment the invention relates to use of a reagent binding a G6PC2-encoded beta cell surface tag to identify or select cells that express a G6PC2-encoded beta cell surface tag as a cell surface marker. In one embodiment the invention relates to use of a G6PC2-encoded beta cell surface tag as a cell surface marker to obtain a culture of pancreatic endocrine cells. In one embodiment one or more further cell surface markers are used simultaneously or sequentially to obtain a culture of pancreatic endocrine cells. In one embodiment a further cell surface marker is selected from the group consisting of DNER protein, DDR1 protein, prominin 1 (also known as CD133), and CD49f. In one embodiment a further cell surface marker is DNER protein or DDR1 protein. In one embodiment the G6PC2-encoded beta cell surface tag is a part of the full-length IGRP.

In one embodiment the invention relates to an antibody that specifically binds to a G6PC2-encoded beta cell surface tag.

In one embodiment the invention said G6PC2-encoded beta cell surface tag is as defined herein.

### DESCRIPTION OF THE INVENTION

It has surprisingly been found that G6PC2-encoded epitopes, optionally splice variants hereof, provide useful markers for fully differentiated beta cells. In one embodiment G6PC2-encoded full length peptides and/or fragments hereof provide useful markers for fully differentiated beta cells. In one embodiment G6PC2-encoded non-full length peptides and optionally splice variants hereof provide useful markers for fully differentiated beta cells. In one embodiment non-full length G6PC2-encoded peptides are derouted to the surface of such cells. In one embodiment the N-terminal sequence of particular splice variants of G6PC2 as defined herein is derouted to the surface of such cells. In one embodiment all known splice variants of G6PC2 share the same N-terminal amino acid sequence as the full length variant which is normally localized towards the lumen of the ER (endoplasmatic reticulum). In one embodiment non-full length splice variants are not retained in the ER and end up at the cell surface plasma membrane where the N-terminal sequence thus serve as purification tag for beta cells.

In one embodiment the isolated peptide is present and/or detectable on the outer surface of a cell. In one embodiment methods described herein, such as methods of identification, can be used to determine whether present and/or detectable on the outer surface of a cell.

### G6PC2

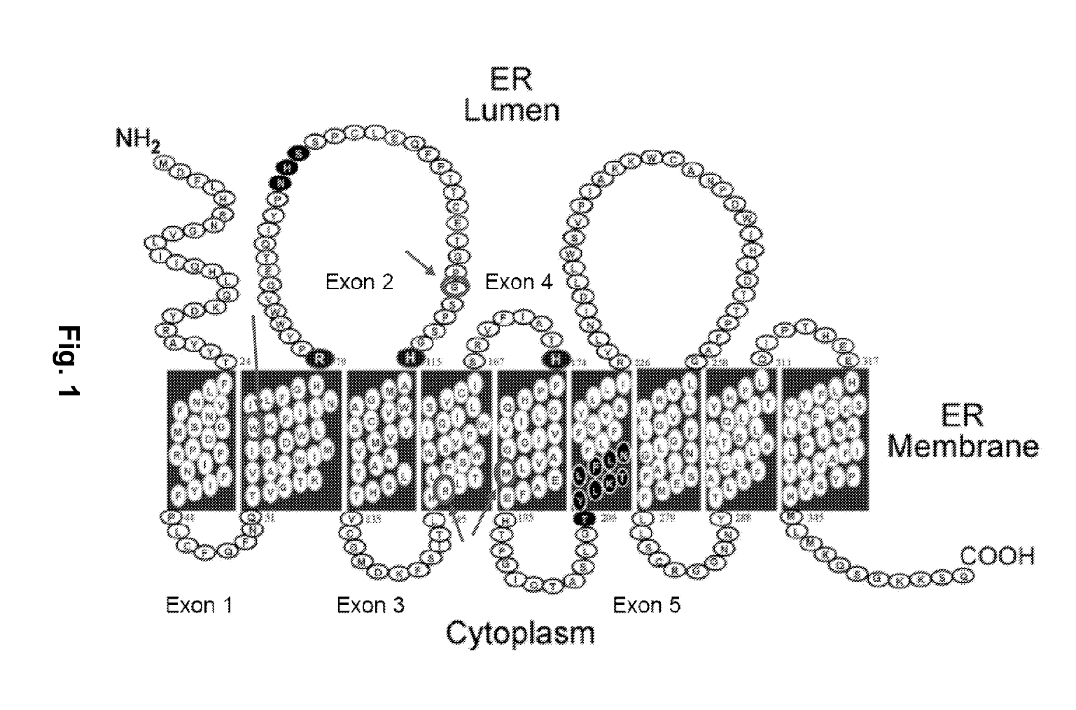

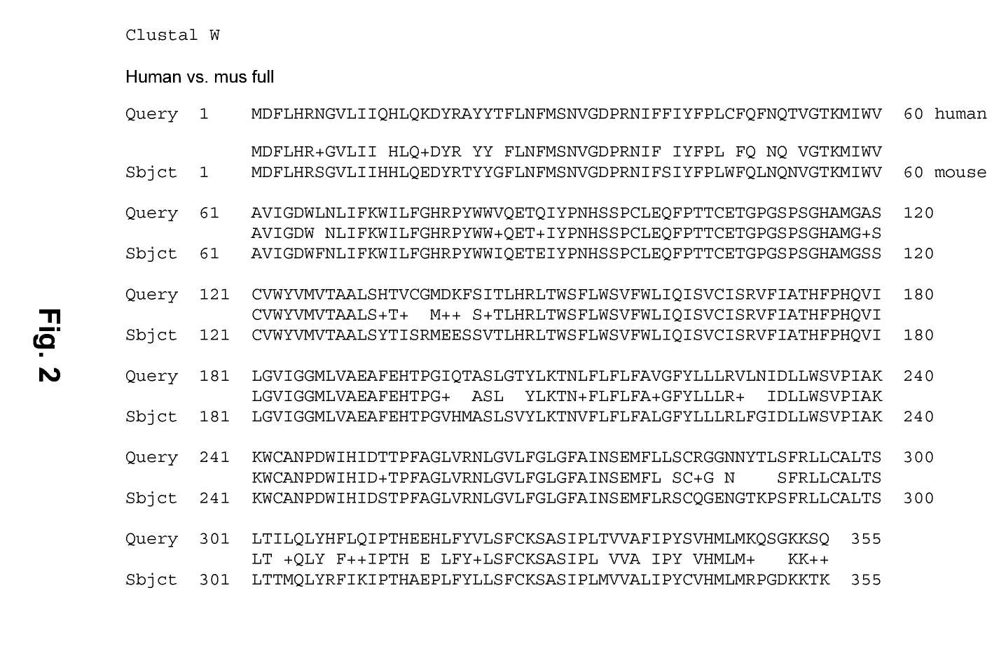

As used herein, “G6PC2-encoded peptide”, “islet-specific Glucose-6-phosphate catalytic-subunit Related Protein” or “IGRP” refers to all mammalian forms of IGRP, including human and mouse. In one embodiment G6PC2 is the human gene encoding IGRP. IGRP has a highly restricted endocrine beta-islet expression. IGRP is also expressed at low levels in the thymus. Full-length IGPR is encoded by 5 exons and is a complex membrane protein of the Endoplasmic Reticulum (ER) with 9 trans-membrane stretches resulting in 5 protein domains oriented towards the ER-lumen, and 5 domains oriented towards the cytoplasm, see FIG. 1. The amino acid sequence of mouse G6PC2 (SEQ ID NO: 2) is shown in FIG. 2. The amino acid sequence of human G6PC2 (SEQ ID NO: 1) is shown below, wherein the underlined amino acid indicates an exon junction.

```
    1 MDFLHRNGVLIIQHLQKDYRAYYTFLNFMSNVGDPRNIFFIYFPLCFQFNQTVGTKMIWV
  61 AVIGDWLNLIFKWILFGHRPYWWVQETQIYPNHSSPCLEQFPTTCETGPGSPSGHAMGAS
121 CVWYVMVTAALSHTVCGMDKFSITLHRLTWSFLWSVFWLIQISVCISRVFIATHFPHQVI
181 LGVIGGMLVAEAFEHTPGIQTASLGTYLKTNLFLFLFAVGFYLLLRVLNIDLLWSVPIAK
241 KWCANPDWIHIDTTPFAGLVRNLGVLFGLGFAINSEMFLLSCRGGNNYTLSFRLLCALTS
301 LTILQLYHFLQIPTHEEHLFYVLSFCKSASIPLTVVAFIPYSVHMLMKQSGKKSQ
```

G6PC2-encoded protein as an endoplasmic reticulum (ER) membrane protein is not considered a candidate for a cell surface marker due to its intracellular localization. However, alternative mRNA processing is likely to lead to loss of the ER retention signal (which may be of undefined character as compared to the classical C-terminal KDEL sequence as a ER retention signal) and such splice variants would then be derouted to the surface.

Novel data disclosed herein demonstrates the identification of hitherto unknown G6PC2-encoded splice variants that are expressed in the islet of Langerhans. Some of these variants are associated with frame shifts leading to novel stretches of amino acids in epitopes as well as to derouting of the shortened protein towards the cell surface where they serve as unique markers for the islet beta cell. In one embodiment the splice variants retain an intact N-terminus. Several splice variants represent substantial structural change, including swap from cytoplasmic to extracellular exposure of particular domains and including loss of ER retention signals and accumulation on the cell surface membrane.

The present inventors have found that N-terminal epitopes as well as beta cell-selective splice variant-generated epitopes of IGRP are novel surface tags for the mature insulin producing cell. In one embodiment said epitopes (also called tags or surface tags) can be used in e.g. antibody-mediated cell purification of mature beta cells from cell preparations (such as human beta cells generated by differentiating hESC) as well as the use of such tags as targets to quantify or image beta cell populations in vitro and in vivo.

In one embodiment antibodies raised against the G6PC2-encoded beta cell surface tag can be used to purify mature beta cells from a mixed cell population. In one embodiment the mixed cell population is derived from pancreatic tissue and/or from endocrine cultures derived from differentiating stem cells.

“G6PC2-encoded beta cell surface tag”, “G6PC2-encoded epitope”, “G6PC2-encoded beta cell surface marker” or “G6PC2-splice variant-encoded beta cell surface tag” as used herein refers to extracellular sequences of IGRP, encoded by G6PC2, or splice variants thereof.

In one embodiment the isolated peptide according to the invention may be used as a G6PC2-encoded beta cell surface tag. In one embodiment the G6PC2-encoded beta cell surface tag is comprised in the isolated peptide according to the invention. In one embodiment the G6PC2-encoded beta cell surface tag is the isolated peptide according to the invention.

In one embodiment the invention relates to an isolated peptide encoded by G6PC2, wherein said peptide comprises i) an extracellular part and ii) a deletion in one or more exons of G6PC2. In one embodiment said peptide comprises an extracellular part. In one embodiment said peptide consists of an extracellular part. In one embodiment said extracellular part comprises an epitope. In one embodiment said isolated peptide comprises a sequence contained in one or more exons selected from the group consisting of exon 1, exon 2, exon 3, exon 4 and exon 5 of G6PC2. In one embodiment said isolated peptide is a fragment of a sequence contained in one exon or several contiguous exons selected from the group consisting of exon 1, exon 2, exon 3, exon 4 and exon 5 of G6PC2. In one embodiment said isolated peptide comprises a deletion in a sequence selected from one or more from the group consisting of exon 1, exon 2, exon 3, exon 4 and exon 5 of G6PC2.

In one embodiment said isolated peptide comprises a consecutive sequence of a G6PC2-encoded splice variant part of the N-terminal end and is truncated in the C-terminal end. In one embodiment said isolated peptide comprises a sequence selected from the group consisting of MDFLHRNGVLIIQHLQKDYRAYYT (SEQ ID NO: 3), MLVAEAFEHTPGIQ (SEQ ID NO: 8), LLSCRGGNNY (SEQ ID NO: 5) and HMLMKQSGKKSQ (SEQ ID NO: 6) as well as variants thereof. In one embodiment said isolated peptide comprises the sequence MLVAEAFEHTPGIQTASLGT (SEQ ID NO: 4) or variants thereof. In one embodiment said isolated peptide consists of a sequence selected from the group consisting of MDFLHRNGVLIIQHLQKDYRAYYT (SEQ ID NO: 3), MLVAEAFEHTPGIQ (SEQ ID NO: 8), LLSCRGGNNY (SEQ ID NO: 5) and HMLMKQSGKKSQ (SEQ ID NO: 6) as well as variants thereof. In one embodiment said isolated peptide consists of the sequence MLVAEAFEHTPGIQTASLGT (SEQ ID NO: 4) or variants thereof. In one embodiment the G6PC2-splice variant-encoded beta cell surface tag is contained in exon 1 of G6PC2. In one embodiment the N-terminal G6PC2-encoded beta cell surface tag is MDFLHRNGVLIIQHLQKDYRAYYT (SEQ ID NO: 3). In one embodiment the G6PC2-splice variant-encoded beta cell surface tag is derived from exons 1, 2 and 5 of G6PC2. In one embodiment the G6PC2-splice variant-encoded beta cell surface tag is selected from the group consisting of MLVAEAFEHTPGIQ (SEQ ID NO: 8), LLSCRGGNNY (SEQ ID NO: 5) and HMLMKQSGKKSQ (SEQ ID NO: 6). In one embodiment the G6PC2-splice variant-encoded beta cell surface tag is MLVAEAFEHTPGIQTASLGT (SEQ ID NO: 4).

In one embodiment said isolated peptide comprises a naturally occurring amino acid sequence of at least 80%, such as at least 90%, or at least 96% identity to an amino acid sequence selected from the group consisting of MDFLHRNGVLIIQHLQKDYRAYYT (SEQ ID NO: 3), MLVAEAFEHTPGIQ (SEQ ID NO: 8), LLSCRGGNNY (SEQ ID NO: 5) and HMLMKQSGKKSQ (SEQ ID NO: 6). In one embodiment said isolated peptide comprises a naturally occurring amino acid sequence of at least 80%, such as at least 90%, or at least 96% identity to the amino acid sequence MLVAEAFEHTPGIQTASLGT (SEQ ID NO: 4). In one embodiment the G6PC2-splice variant-encoded beta cell surface tag consists of at least 4, such as at least 6, 8, 10, 12, 14 or 16 amino acids.

In one embodiment the G6PC2-encoded beta cell surface tag is a part of the full-length IGRP. In one embodiment the G6PC2-encoded beta cell surface tag is a part of a non full-length splice variant of IGRP. In one embodiment the G6PC2-encoded beta cell surface tag is a non full-length splice variant of IGRP. In one embodiment the G6PC2-encoded beta cell surface tag is a fragment of a sequence contained in one exon or several contiguous exons selected from the group consisting of exon 1, exon 2, exon 3, exon 4 and exon 5 of G6PC2.

In one embodiment the invention relates to an isolated nucleotide encoding a peptide as defined herein. In one embodiment the invention relates to an isolated cell expressing a peptide as defined herein. In one embodiment the G6PC2-encoded beta cell surface tag comprises an extracellular sequence. In one embodiment the G6PC2-encoded beta cell surface tag consists of an extracellular sequence.

In one embodiment the G6PC2-encoded beta cell surface tag is a fragment of the isolated peptide as defined herein. In one embodiment the G6PC2-encoded beta cell surface tag is a part of the full-length IGRP. In one embodiment the invention relates to an isolated nucleotide encoding a G6PC2-encoded beta cell surface tag as defined herein. In one embodiment the invention relates to an isolated cell expressing a G6PC2-encoded beta cell surface tag as defined herein.

“Splice variant” as used herein refers to RNA splicing variation mechanism in which the exons of the primary gene transcript, the pre-mRNA, are separated and reconnected so as to produce alternative ribonucleotide arrangements. These linear combinations then undergo the process of translation where specific and unique sequences of amino acids are specified, resulting in isoform proteins. “Fragment of” as used herein refers to a sequence that is shorter than the sequence referred to. “Part of” or “non full-length” as used herein refers to a sequence that contained in the larger sequence referred to.

In one embodiment the terms “peptide” and “amino acid sequence” refer to an oligopeptide, a peptide, a polypeptide, or a protein sequence, or a fragment of any of these. In one embodiment the terms “amino acid” and “amino acid sequence” refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

In one embodiment a “variant” of a particular nucleic acid sequence is defined as a nucleic acid sequence having at least 40% sequence identity to the particular nucleic acid sequence over a certain length of one of the nucleic acid sequences. In one embodiment a “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0. 9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A splice variant may have significant identity to a reference molecule, but will generally have a greater or lesser number of polynucleotides due to alternate splicing during mRNA processing. The corresponding polypeptide may possess additional functional domains or lack domains that are present in the reference molecule. In one embodiment a “variant” of a particular polypeptide sequence is defined as a polypeptide sequence having at least 40% sequence identity or sequence similarity to the particular polypeptide sequence over a certain length of one of the polypeptide sequences. In one embodiment a “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

In one embodiment “identity” or “sequence identity” is determined over the entire length of the sequence to which comparison is made, i.e. the reference sequence. As an example of a method for determination of sequence identity between two sequences QBCDE and ABCDE are aligned. The sequence identity of QBCDE relative to ABCDE is given by the number of aligned identical residues minus the number of different residues divided by the total number of residues in ABCDE. Accordingly, in said example the sequence identity is (5−1)/5.

In one embodiment the phrases “percent identity” and “% identity” as applied to polypeptide sequences, refer to the percentage of identical residue matches between at least two polypeptide sequences aligned using a standardized algorithm. Methods of polypeptide sequence alignment are well-known. Some alignment methods take into account conservetive amino acid substitutions. Such conservative substitutions generally preserve the charge and hydrophobicity at the site of substitution, thus preserving the structure (and therefore function) of the polypeptide. In one embodiment the phrases “percent similarity” and “% similarity”, as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

In one embodiment percent identity, such as sequence identity, between polypeptide sequences may be determined using the default parameters of the CLUSTAL V algorithm as incorporated into the MEGALIGN version 3.12e sequence alignment program. For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. In one embodiment the NCBI BLAST software suite may be used for determination of percent identity, such as sequence identity. For example, for a pairwise comparison of two polypeptide sequences, one may use the “BLAST 2 Sequences” tool Version 2.0. 12 (Apr. 21, 2000) with blastp set at default parameters; such default parameters may be, for example: Matrix: BLOSUM62; Open Gap: 11 and Extension Gap: I penalties; Gap x drop-off.-50; Expect: 10; Word Size: 3; Filter: on.

In one embodiment percent identity, such as sequence identity, may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

In one embodiment the invention does not relate to MDFLHRNGVLIIQHLQKDYRAYYTFLNFMSNVGDPRNIFFIYFPLCFQFNQTVGTKMIWVAVIGDWLNLIFKWKSIWPCNGRILCLVCHGNRCPEPHCLWDG or SEQ ID No. 38 as defined in WO2004001008.

Pancreatic Endocrine Cells—and their Progenitors

In the pancreas several different types of pancreatic cells may be found. These cells include for example multi-potent pancreatic progenitor cells, ductal/endocrine progenitor cells, endocrine pre-progenitor cells, endocrine progenitor cells, early endocrine cells, fully differentiated endocrine cells and/or fully differentiated beta cells.

“Pancreatic endocrine cell” or “pancreatic hormone expressing cell” as used interchangeably herein refers to a cell capable of expressing at least one of the following hormones: insulin, glucagon, somatostatin, pancreatic polypeptide, and ghrelin.

“Pancreatic hormone secreting cell” as used herein refers to a pancreatic endocrine cell capable of secreting at least one of the following hormones: insulin, glucagon, somatostatin, pancreatic polypeptide, and ghrelin.

“Pancreatic endocrine pre-progenitor cells” (also termed “pancreatic endocrine preprogenitors” and “endocrine pre-progenitors”) as used herein are cells, which have lost their potential of developing into pancreatic ductal and exocrine cells, have not yet expressed Ngn3 protein, and are not hormone expressing, but which have the potential to differentiate into pancreatic endocrine cells or pancreatic hormone secreting cells, and which do normally also share at least part of the phenotype characteristic of these cells.

“Pancreatic endocrine progenitor cells” (also termed “pancreatic endocrine progenitors” and “endocrine progenitors”) as used herein are cells, which are Ngn3 protein expressing cells but not hormone expressing, but which have the potential to differentiate into pancreatic endocrine cells or pancreatic hormone secreting cells, and which do normally also share at least part of the phenotype characteristic of these cells.

“Early endocrine cells” (also termed “pancreatic early endocrine cells”) as used herein are endocrine cells which have turned off Ngn3 but do not share all the characteristics of fully differentiated pancreatic endocrine cells found in the islet of Langerhans in the adult pancreas, such as responsiveness to glucose. The early endocrine cells may be negative or positive for one of the pancreatic endocrine hormones (insulin, glucagon, somatostatin, pancreatic polypeptide, and ghrelin).

“Fully differentiated endocrine cells” (also termed “pancreatic mature endocrine cells”) as used herein are cells which share all the characteristics of fully differentiated pancreatic endocrine cells found in the islet of Langerhans in the adult pancreas. “Pancreatic hormone expressing cells” and “pancreatic hormone secreting cells” are considered as “pancreatic endocrine cells” ranging from the early to the fully differentiated phenotype.—

“Fully differentiated beta cells” (also termed “mature beta cells”) as used herein are glucose sensitive insulin producing beta cells which characterize the major cell type constituting the islet of Langerhans in the adult pancreas.

“beta cell lineage” as used herein refer to cells with positive gene expression for the transcription factor Pdx-1 and at least one of the following transcription factors: Ngn-3, Nkx2.2, Nkx6.1, NeuroD, Isl-1, Hnf-3 beta, MafA, Pax4, and Pax6. Cells expressing markers characteristic of the beta cell lineage include beta cells.

“Ductal/endocrine progenitor cells” as used herein are cells which during early pancreas development reside in the central part and retain the bi-potential of becoming mature ductal cells, fully differentiated endocrine cells or fully differentiated beta cells. Furthermore, these cells express the transcription factors Pdx1 and Nkx6.1 and not Ptf1a. An example of ductal/endocrine progenitor cells can be found at development stage around e12.0 in the mouse.

“Multi-potent pancreatic progenitor cells” as used herein are cells which represents the earliest cells for the pancreas. These cells are uniquely characterized as the triple positive cells for the 3 key transcription factors: Pdx1⁺/Nkx6.1⁺/Ptf1a⁺.

“Markers” as used herein, are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

In one embodiment the pancreatic endocrine cells obtained by the method according to the invention are insulin producing cells, optionally together with cells differentiated towards glucagon, somatostatin, pancreatic polypeptide, and/or ghrelin producing cells. As used herein, “insulin producing cells” refers to cells that produce and store or secrete detectable amounts of insulin. “Insulin producing cells” can be individual cells or collections of cells.

“Passage” of cells usually refers to a transition of a seeded culture container from a partially confluent state to a confluent state, at which point they are removed from the culture container and reseeded in a culture container at a lower density. However, cells may be passaged prior to reaching confluence. Passage typically results in expansion of the cell population as they grow to reach confluence. The expansion of the cell population depends on the initial seeding density but is typically a 1 to 10, 1 to 5, 1 to 3, or 1 to 2 fold expansion. Thus, passaging generally requires that the cells be capable of a plurality of cell divisions in culture.

### Cell Population Comprising Pancreatic Cells

The term “a cell population comprising pancreatic cells” as used herein refers to a population of cells comprising one or more cell types selected from the group consisting of acinar and ductal cells, multi-potent pancreatic progenitor cells, ductal/endocrine progenitor cells, pancreatic endocrine pre-progenitor cells, pancreatic endocrine progenitor cells, early pancreatic endocrine cells, pancreatic endocrine cells, pancreatic beta cells, pancreatic hormone secreting cells, fetal pancreatic cells, adult pancreatic cells and other non-pancreatic cells. A “population” of cells refers to a plurality of cells obtained by a particular isolation of the starting cells or culture procedure. Properties of a cell population are generally defined by a percentage of individual cells having the particular property (e.g. the percentage of cells staining positive for a particular marker) or the bulk average value of the property when measured over the entire population (e.g. the amount of mRNA in a lysate made from a cell population, or percentage of cells positive for a histochemically detectable marker, such as Ngn3, Pax6, insulin or glucagon).

In one embodiment the cell population comprising pancreatic cells is obtained from a pancreas. In one embodiment the cell population comprising pancreatic cells is obtained from a fetal pancreas or an adult pancreas. In one embodiment the pancreas is from a mammal, such as a human.

In one embodiment of the invention, the cell population comprising pancreatic cells is obtained from a somatic cell population. In one embodiment of the invention, the somatic cell population has been induced to de-differentiate in to an embryonic-like stem (ES, e.g. a pluripotent) cell. Such de-differentiated cells are also termed induced pluripotent stem cells (IPS).

In one embodiment the cell population comprising pancreatic cells is obtained from embryonic stem (ES, e.g. pluripotent) cells. In one embodiment the cell population comprising pancreatic cells is pluripotent cells, such as ES cells. In one embodiment the cell population comprising pancreatic cells is pluripotent cells, such as ES like-cells.

In one embodiment the cell population comprising pancreatic cells is embryonic differentiated stem (ES or pluripotent) cells. Differentiation takes place in embryoid bodies and/or in monolayer cell cultures or a combination thereof.

In one embodiment the cell population comprising pancreatic cells is of mammalian origin. In one embodiment the cell population comprising pancreatic cells is of human origin. In one embodiment of the invention, the cell population has been differentiated to the pancreatic endocrine lineage.

In one embodiment the cell population comprising pancreatic cells is obtained from one or more donated pancreases. The methods described herein are not dependent on the age of the donated pancreas. Accordingly, pancreatic material isolated from donors ranging in age from embryos to adults can be used.

Once a pancreas is harvested from a donor, it is typically processed to yield individual cells or small groups of cells for culturing using a variety of methods. One such method calls for the harvested pancreatic tissue to be cleaned and prepared for enzymatic digestion. Enzymatic processing is used to digest the connective tissue so that the parenchyma of the harvested tissue is dissociated into smaller units of pancreatic cellular material. The harvested pancreatic tissue is treated with one or more enzymes to separate pancreatic cellular material, substructures, and individual pancreatic cells from the overall structure of the harvested organ. Collagenase, DNAse, Liberase preparations (see U.S. Pat. Nos. 5,830,741 and 5,753,485) and other enzymes are contemplated for use with the methods disclosed herein.

Isolated source material can be further processed to enrich for one or more desired cell populations. However, unfractionated pancreatic tissue, once dissociated for culture, can also be used directly in the culture methods of the invention without further separation. In one embodiment the isolated pancreatic cellular material is purified by centrifugation through a density gradient (e.g., Nycodenz, Ficoll, or Percoll). For example the gradient method described in U.S. Pat. No. 5,739,033, can be used as a means for enriching the processed pancreatic material in islets. The mixture of cells harvested from the donor source will typically be heterogeneous and thus contain alpha-cells, beta-cells, delta-cells, epsilon-cells, ductal cells, acinar cells, facultative progenitor cells, and other pancreatic cell types.

A typical purification procedure results in the separation of the isolated cellular material into a number of layers or interfaces. Typically, two interfaces are formed. The upper interface is islet-enriched and typically contains 10 to 100% islet cells in suspension. The second interface is typically a mixed population of cells containing islets, acinar, and ductal cells. The bottom layer is the pellet, which is formed at the bottom of the gradient. This layer typically contains primarily acinar cells, some entrapped islets, and some ductal cells. Ductal tree components can be collected separately for further manipulation. The cellular constituency of the fractions selected for further manipulation will vary depending on which fraction of the gradient is selected and the final result of each isolation.

When islet cells are the desired cell type, a suitably enriched population of islet cells within an isolated fraction will contain at least 10% to 100% islet cells. Other pancreatic cell types and concentrations can also be harvested following enrichment. For example, the culture methods described herein can be used with cells isolated from the second interface, from the pellet, or from other fractions, depending on the purification gradient used.

In one embodiment intermediate pancreatic cell cultures are generated from the islet-enriched (upper) fraction. Additionally, however, the more heterogeneous second interface and the bottom layer fractions that typically contain mixed cell populations of islets, acinar, and ductal cells or ductal tree components, acinar cells, and some entrapped islet cells, respectively, can also be used in culture. While both layers contain cells capable of giving rise to the G6PC2 positive population described herein, each layer may have particular advantages for use with the disclosed methods. In one embodiment G6PC2 positive pancreatic cell cultures are generated from the islet-enriched (upper) fraction.

In one embodiment the cell population is a population of stem cells. In one embodiment of the invention, the cell population is a population of stem cells differentiated to the pancreatic endocrine lineage.

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multi-potent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multi-potent stem cells; and (5) unipotent, meaning able to give rise to a single cell lineage (e.g., spermatogenic stem cells).

A protocol for obtaining pancreatic cells from stem cells is exemplified by, but not limited to, the protocols described in D'Amour, K. A. et al. (2006), Nat Biotechnol 24, 1392-401; Jiang, J. et al. (2007), Stem Cells 25, 1940-53; and Kroon, E. et al. (2008), Nat Biotechnol, 2008 Feb. 20, [Epub ahead of print].

A protocol for obtaining pancreatic cells from somatic cells or somatic cells induced to de-differentiate into pluripotent cells, such as ES like-cells is exemplified by, but not limited to, the protocols described in Aoi, T. et al. (2008), Science, 2008 Feb. 14, [Epub ahead of print]; D'Amour, K. A. et al. (2006), Nat Biotechnol 24, 1392-401; Jiang, J. et al. (2007), Stem Cells 25, 1940-53; Kroon, E. et al. (2008), Nat Biotechnol, 2008 Feb. 20, [Epub ahead of print]; Takahashi, K. et al. (2007), Cell 131, 861-72; Takahashi, K., and Yamanaka, S. (2006), Cell 126, 663-76; and Wernig, M. et al. (2007), Nature 448, 318-24.

Differentiation is the process by which an unspecialized (“uncommitted”) or less specialized cell acquires the features of a specialized cell, such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. Dedifferentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein “differentiate” or “differentiation” refers to a process where cells progress from an undifferentiated state to a differentiated state, from an immature state to a less immature state or from an immature state to a mature state. For example, early undifferentiated embryonic pancreatic cells are able to proliferate and express characteristics markers, like Pdx-1, Nkx6.1, and Ptf1a. Mature or differentiated pancreatic cells do not proliferate and do secrete high levels of pancreatic endocrine hormones or digestive enzymes. E.g., fully differentiated beta-cells secrete insulin at high levels in response to glucose. Changes in cell interaction and maturation occur as cells lose markers of undifferentiated cells or gain markers of differentiated cells. Loss or gain of a single marker can indicate that a cell has “matured or fully differentiated.” The term “differentiation factors” refers to a compound added to pancreatic cells to enhance their differentiation to mature endocrine cells also containing insulin producing beta cells. Exemplary differentiation factors include hepatocyte growth factor, keratinocyte growth factor, exendin-4, basic fibroblast growth factor, insulin-like growth factor-1, nerve growth factor, epidermal growth factor platelet-derived growth factor, and glucagon-like-peptide 1. In one embodiment differentiation of the cells comprises culturing the cells in a medium comprising one or more differentiation factors.

Markers characteristic of the pancreatic endocrine lineage are selected from the group consisting of Ngn 3, NeuroD, islet-1, Pdx-1, Nkx6.1, Nkx2.2, MafA, MafB, Arx, Brn4, Pax-4, Pax-6, Glut2, insulin, glucagon, somatostatin, pancreatic polypeptide (PP), and ghrelin. In one embodiment a pancreatic endocrine cell is capable of expressing at least one of the following hormones: insulin, glucagon, somatostatin, PP, and ghrelin. Suitable for use in the present invention is a cell that expresses at least one of the markers characteristic of the pancreatic endocrine lineage. In one embodiment of the present invention, a cell expressing markers characteristic of the pancreatic endocrine lineage is a pancreatic endocrine cell. The pancreatic endocrine cell may be a pancreatic hormone expressing cell. Alternatively, the pancreatic endocrine cell may be a pancreatic hormone secreting cell.

In one embodiment of the present invention, the pancreatic endocrine cell is a cell expressing markers characteristic of the beta cell lineage. A cell expressing markers characteristic of the beta cell lineage expresses Pdx1 and at least one of the following transcription factors: Ngn-3, Nkx2.2, Nkx6.1, NeuroD, Isl-1, Hnf-3 beta, MafA, Pax4, and Pax6. In one embodiment of the present invention, a cell expressing markers characteristic of the beta cell lineage is a beta cell. In one embodiment the pancreatic endocrine cell is a cell expressing the marker Nkx6.1. In one embodiment of the invention, the pancreatic endocrine cell is a cell expressing the marker Pdx1. In one embodiment of the invention, the pancreatic endocrine cell is a cell expressing the markers Nkx6.1 and Pdx1.

“Pdx-1” as used herein refers to a homeodomain transcription factor implicated in pancreas development. “Pax-4” as used herein is a beta cell specific transcription factor and “Pax-6” as used herein is a pancreatic islet cell (specific) transcription factor; both are implicated in islet development. “Hnf-3 beta” belong to the hepatic nuclear factor family of transcription factors, which is characterized by a highly conserved DNA binding domain and two short carboxy-terminal domains. “NeuroD” as used herein is basic helix-loop-helix (bHLH) transcription factor implicated in neurogenesis. “Ngn-3” as used herein, is a member of the neurogenin family of basic loop-helix-loop transcription factors. “Nkx-2.2” and “Nkx-6.1” as used herein are members of the Nkx transcription factor family. “islet-1” or “Isl-1” as used herein is a member of the LIM/homeodomain family of transcription factors, and is expressed in the developing pancreas. “MafA” as used herein is a transcription factor expressed in the pancreas, and controls the expression of genes involved in insulin biosynthesis and secretion.

Nkx6.1 and Pdx-1 are co-expressed with Ptf1a in the early pancreatic multi-potent cell that can develop into all cell types found in the adult pancreas (e.g., acinar, ductal, and endocrine cells). Within this cell population cells that also transiently express Ngn3 are found. Once a cell express or has expressed Ngn3 it will be part of the endocrine lineage, giving rise to endocrine cells (one type being the insulin producing beta cell) that will later form the islets of Langerhans. In the absence of Ngn3 no endocrine cells form during pancreas development. As development progress Nkx6.1 and Pdx-1 are co-expressed in the more central domain of the pancreas which now become devoid of Ptf1a expression and the Nkx6.1 and Pdx-1 positive cells can no longer give rise to acinar cells. Within this Nkx6.1 and Pdx-1 positive cell population a significant number of cells transiently co-express Ngn3, marking them for the endocrine lineage like earlier in development.

### DNER

As used herein, “DNER”, “Dner”, “DNER protein”, or “Dner protein” refers to all mammalian forms of DNER, including human and mouse. The human form is known as: “delta/notch-like EGF repeat containing”, also known as, e.g., “bet” and “UNQ26”. The mouse form is known as: “delta/notch-like EGF-related receptor” also known as, e.g., “BET”, “Bret”, “MGC39059”, and “A930026D19Rik”. DNER contains a single transmembrane domain at its C-terminal end and is presumed to be a putative cell surface protein. It also contains a number EGF-like repeats in its extracellular domain and its cytoplasmic carboxy-terminal domain contains a tyrosine-based sorting motif. In both human and mouse it is 737 amino acids long, and the similarity between mouse and human is 90%. One family member has been identified in mouse. Dner acts as a ligand of Notch during cellular morphogenesis of Bergmann glia in the mouse cerebellum. Dner binds to Notch1 at cell-cell contacts and activate Notch signalling in vitro.

### DDR1

As used herein, “DDR1 protein” or “Ddr1 protein” refers to a DDR/TKT type protein kinase, Discoidin Domain Receptor family, member 1. When used herein the term may be written fully in uppercase, “DDR1”, or with only the first letter in uppercase, “Ddr1”, and shall mean the Discoidin Domain Receptor family, member 1 from any mammal including human and mouse. DDR1 is activated by various types of collagen, including types I through IV. Binding of collagen to DDR1 protein results in autophosphorylation and a delayed but sustained tyrosine kinase activation. DDR1 may function in cell-to-cell interaction or recognition. At least three mRNA variants, resulting in different protein isoforms of 876, 913 and 919 amino acids, have been reported in humans. In the mouse two isoforms have been reported of 874 and 911 amino acids, respectively. DDR1 protein has been shown to be overexpressed in human breast, ovarian, esophageal and pediatric brain tumors. The protein has an intracellular Receptor tyrosine kinases activity and is activated by various types of collagen. Its autophosphorylation is achieved by all collagens so far tested (type Ito type VI and XI).

### Methods of Identification

In one embodiment the invention relates to a method of identification of fully differentiated beta cells, the method comprising contacting a cell population comprising pancreatic cells with a reagent binding G6PC2-encoded beta cell surface tag. In one embodiment the number and/or ratio of cells that binds the reagent binding G6PC2-encoded beta cell surface tag, i.e. cells positive for G6PC2-encoded beta cell surface tag, may be determined.

In one embodiment the invention relates to a method of quantifying cells positive for G6PC2-encoded beta cell surface tag comprising pancreatic cells by a) contacting the cells with a reagent binding G6PC2-encoded beta cell surface tag; and b) determining the quantity of cells that exhibit G6PC2-encoded beta cell surface tag as a cell surface marker (cells positive for G6PC2-encoded beta cell surface tag).

Those skilled in the art will recognize that there are many methods to detect a G6PC2-encoded beta cell surface tag. For example, antibodies that bind specifically to the G6PC2-encoded beta cell surface tag can be used to detect the G6PC2-encoded beta cell surface tag. Antibodies specific to the G6PC2-encoded beta cell surface tag are known to those skilled in the art and are commercially available from, for example, R&D Systems, Research Diagnostics, Inc.; Abcam; Ancell Immunology Research Products; eBioscience; the Developmental Studies Hybridoma Bank of the Univeristy of Iowa; and Zymed Laboratories, Inc., Abnova Corporation, -Affinity BioReagents BioLegend, GeneTex Lifespan Biosciences, MBL International Novus Biologicals, Proteintech Group, Inc., Santa Cruz Biotechnology, Inc. Antibodies that recognize the extracellular portion of a G6PC2-encoded beta cell surface tag may be used in the present invention for sorting cells. Different antibodies that recognize different epitopes on the extracellular portion of G6PC2-encoded beta cell surface tag may be used either alone or in combination. Any reagent binding G6PC2-encoded beta cell surface tags that recognize any part of G6PC2-encoded beta cell surface tag both in the extracellular domain, transmembrane domain and intracellular domain can be used for monitoring expression of G6PC2-encoded beta cell surface tag. A person skilled in the art would realise that the description herein of detection of G6PC2-encoded beta cell surface tag would also apply to other extracellular proteins which may be contemplated for use as markers in the present invention.

Many different fluorescent molecules are available for conjugation to antibodies, for example fluorescien, cy2, cy3, cy5, PE, Alexa488, or rhodamine. Those skilled are aware that in some instances more than one extracellular marker can be detected by using different antibodies conjugated to fluorescent molecules. FACS-analysis can be done under conditions to identify more than one extracellular marker of interest.

In one embodiment the reagent binding a G6PC2-encoded beta cell surface tag is an antibody that specifically binds to a G6PC2-encoded beta cell surface tag.

In one embodiment the invention relates to an antibody that specifically binds to a G6PC2-encoded beta cell surface tag. In one embodiment the G6PC2-encoded beta cell surface tag is a part of the full-length IGRP.

“Antibody” or “antibodies” refer to a polypeptide comprising a framework region from an immunoglobulin gene or fragments thereof that specifically binds and recognizes an antigen. Antibodies (also known as immunoglobulins) are proteins that are found in blood or other bodily fluids of vertebrates, and are used by the immune system to identify and neutralize foreign objects, such as bacteria and viruses. Although the general structure of all anti-bodies is very similar, a small region at the tip of the protein is extremely variable, allowing millions of antibodies with slightly different tip structures to exist. This region is known as the hypervariable region. Each of these variants can bind to a different target, known as an antigen. This huge diversity of antibodies allows the immune system to recognize an equally wide diversity of antigens. The unique part of the antigen recognized by an antibody is called an epitope. These epitopes bind with their antibody in a highly specific interaction, called induced fit, that allows antibodies to identify and bind only their unique antigen in the midst of the millions of different molecules that make up an organism.

Methods for assessing expression of protein and/or mRNA in cultured or isolated cells are standard in the art and include quantitative reverse transcription polymerase chain reaction (RT-PCR), Northern blots, and in situ hybridization (see, e.g., Current Protocols in Molecular Biology (Ausubel et al., eds. 2001 supplement)) and immunoassays, such as immunohistochemical analysis of sectioned material, Western blotting, and, for markers that are accessible in intact cells, flow cytometry analysis (FACS) (see, e.g., Harlow and Lane, Using Antibodies: A Laboratory Manual, New York: Cold Spring Harbor Laboratory Press (1998)). Conventional histochemical markers of endocrine cell differentiation may also be employed. Cells to be examined by immunohistochemistry may be cultured on glass chamber slides for microscopic examination. Alternatively, cells grown in conventional tissue culture may be manually, enzymatically or by use of enzyme free cell dissociation buffers removed from the culture and embedded in paraffin or TissueTech for sectioning. Cell differentiation markers are varied and can be detected by conventional immunohistochemistry. A generally applicable protocol follows.

Cells in chamber slides were very gently rinsed with in PBS and fixed for 45 minutes in 4% paraformaldehyde solution. Cells were then rinsed in PBS and stored at +5 until use. At the day of use cells are permeabilized through a graded series of ethanol (starting with 70% moving to 96%, then to 99%, then again 99%, then to 96%, and finally to 70%, using 5 minutes incubation with each concentration) then incubated in a blocking solution containing normal serum or TNB (from the TSA (Tyramide Signal Amplification) kit from Perkin Elmer) at room temperature. Primary antibodies are prepared at appropriate dilution and added to cells and incubated overnight (O/N) at room temperature (RT) in a moist chamber. Following incubation with primary antibody, cells were rinsed in PBS. Fluorescent secondary antibody prepared at appropriate dilution is added to the cells and incubated in the dark. With the secondary antibody DAPI dye might be included for counterstain of cell nuclei. Cells are then rinsed and excess fluid is removed and the chamber portion of the slides removed and slides are mounted with cover slides. The slides dry and are stored in the dark until inspection using a fluorescence microscope, such as a confocal microscope.

Alternatively, the cells can be prepared for immunocytochemistry using the HRP (horse-radish peroxidise) method. As secondary antibody a biotin coupled one is used.

Slides were then rinsed with PBS and incubated with Avidin-HRP. Slides are again rinsed and incubated with TSA reagent to visualize primary antibody. Slides are mounted for visual inspection using conventional light microscopy.

For the identification of proteins in tissue sections the tissue is fixed in 4% PFA (paraformaldehyde) 0/N, then cryo-protected in 30% sucrose 0/N and imbedded in TissueTech. Sections are then cut on a microtome, rinsed in PBS (phosphate buffered saline) and microwaved in 0.01 M citrate buffer (pH 6.0) for 15 minutes to recover epitopes. Such sections can then be stained using either method described above or herein, but omitting the graded ethanol treatment. A hydrogen peroxide solution was used to inhibit endogenous peroxidase activity in the case of using the HRP based assay.

Analytical FACS sorting is carried out on cells that have been fixed for 45 minutes in Lillys fixative. To remove supernatant cells are pelleted by 1400 rpm in 10 minutes at RT in 2 ml tubes. Following this cells are washed in PBS with 0.1% BSA and cells are blocked by adding serum to reach 10% serum (final concentration) from the animal where the secondary antibody is raised, block for 1 h. Cells are then pelleted and supernatant removed. Primary antibody solution is added and incubated O/N at RT. The next day cells are washed 3×5 minutes in 2 ml tubes (using 1.8 ml). Secondary antibody is added and incubated for 1 hour at RT. Cells are washed 3×5 min in PBS with 0.1% BSA (using 1.8 ml). Finally, cells are assayed by FACS.

Identification of fully differentiated beta cells may be achieved by contacting the cell population with a reagent binding G6PC2-encoded beta cell surface tag and evaluating the staining. This analysis may be carried out using a method, such as fluorescence activated cell sorting (FACS), magnetic cell sorting (MACS), immunohistochemistry (IHC), western blot, PCR, or ELISA.

In one embodiment the invention relates to an isolated fully differentiated beta cells obtained by a method as defined herein.

In one embodiment the invention relates to the use of a reagent binding G6PC2-encoded beta cell surface tag to identify or select cells that express G6PC2-encoded beta cell surface tag as a cell surface marker. In one embodiment the invention relates to the use of G6PC2-encoded beta cell surface tag as a cell surface marker to obtain a culture of pancreatic endocrine cells. In one embodiment one or more additional binding reagents are used either simultaneously or sequentially in combination with the reagent binding a G6PC2-encoded beta cell surface tag. In one embodiment said additional binding reagent is selected from the group consisting of DNER, DDR1, prominin 1 (also known as CD133), and CD49f binding reagents. In one embodiment one or more further cell surface markers are used simultaneously or sequentially to obtain a culture of pancreatic endocrine cells. In one embodiment a further cell surface marker is selected from the group consisting of DNER protein, DDR1 protein, prominin 1 (also known as CD133), and CD49f. In one embodiment a further cell surface marker is DNER protein or DDR1 protein.

### Methods of Separating

In one embodiment the invention relates to a method of obtaining a culture of fully differentiated beta cells, the method comprising: contacting a cell population comprising pancreatic cells with a reagent binding G6PC2-encoded beta cell surface tag and separating the cells that binds the reagent binding G6PC2-encoded beta cell surface tag in a fraction of cells positive for G6PC2-encoded beta cell surface tag from cells that do not bind the reagent binding G6PC2-encoded beta cell surface tag.

In one embodiment the step of separating is done by fluorescence activated cell sorting (FACS). In one embodiment the step of monitoring is done by FACS. In one embodiment the step of separating is done by panning. In one embodiment the step of separating is done by MACS.

Fluorescently labelled molecules that bind specifically to G6PC2-encoded beta cell surface tag, most commonly antibodies, are used to select cells positive for G6PC2-encoded beta cell surface tag in conjunction with a Fluorescence Activated Cell Sorter (FACS). Briefly, in one embodiment a cell population comprising pancreatic cells are incubated with fluorescently labelled antibody and after the antibody binding, the cells are analyzed by FACS. The cell sorter passes single cells suspended in liquid through a fluorimeter. The amount of fluorescence is measured and cells with fluorescence levels detectably higher than control, unlabeled, cells are selected as positive cells.

Magnetic cell sorting (MACS) is carried out by incubating specific antibodies bound to G6PC2 on beta cells with iron-particle labelled secondary antibodies, and sequentially sorted using a magnet.

In the embodiment wherein the cell population comprising pancreatic cells are isolated from pancreas, the cells are first cultured for one or more passages and then labelled with an antibody specific for G6PC2-encoded beta cell surface tag. The cells are then scanned using FACS to separate cells positive for G6PC2-encoded beta cell surface tag from cells negative for G6PC2-encoded beta cell surface tag. While this example has discussed FACS analysis with labelled antibodies, other molecules that specifically bind to G6PC2-encoded beta cell surface tag, e.g., lectins and collagens and other binding partners for G6PC2-encoded beta cell surface tag, such as those listed above or herein, can also be used to practice the invention.

In one embodiment the method of separating cells positive for G6PC2-encoded beta cell surface tag from cells negative for G6PC2-encoded beta cell surface tag is by affinity adsorbing cells positive for G6PC2-encoded beta cell surface tag onto a solid support.

Cells positive for G6PC2-encoded beta cell surface tag can also be separated from cells negative for G6PC2-encoded beta cell surface tag by using binding molecules specific for G6PC2-encoded beta cell surface tag, where the binding molecules are attached to a solid support. Those skilled in the art will recognize that molecules specific for G6PC2-encoded beta cell surface tag can be bound to a solid support through an antibody binding molecule, such as protein G or protein A or alternatively, can be conjugated to a solid support directly. Solid supports with attached antibodies against G6PC2-encoded beta cell surface tag are commercially available, e.g., StemSep and EasySep™, magnetic beads, both from Stem Cell Technologies.

Cells positive for G6PC2-encoded beta cell surface tag can also be separated from cells negative for G6PC2-encoded beta cell surface tag through the technique of panning. Panning is done by coating a solid surface with a reagent binding G6PC2-encoded beta cell surface tag and incubating pancreatic cells on the surface for a suitable time under suitable conditions. A flat surface, e.g., a culture dish, is coated with a reagent binding G6PC2-encoded beta cell surface tag.

Pancreatic cells are added to the surface and allowed to bind to the reagent binding G6PC2-encoded beta cell surface tag.

The culture dishes are then washed, removing the cells negative for G6PC2-encoded beta cell surface tag from the dish. In one embodiment an antibody specific for G6PC2-encoded beta cell surface tag is used to coat a culture dish and “pan” for cells positive for G6PC2-encoded beta cell surface tag in a population of pancreatic cells.

In one embodiment the cells may be purified before or after selection by G6PC2-encoded beta cell surface tag by separating cells into Ptprn/IA2-positive and Ptprn-negative cells. In one embodiment the cells may be separated into Abcc8/Sur1-positive and Abcc8/Sur1-negative cells. In one embodiment the cells may be separated into Slc30a8/ZnT-8-positive and Slc30a8/ZnT-8-negative cells. In one embodiment the cells may be purified before or after selection by G6PC2-encoded beta cell surface tag in combination with one or more further extracellular markers, such as DNER protein or DDR1 protein. In one embodiment the cell population comprising pancreatic cells is a beta cell-positive fraction. In one embodiment the cell population comprising pancreatic cells is a ptprn/IA2-positive fraction. In one embodiment the cell population comprising pancreatic cells is an Abcc8/Sur1-positive fraction. In one embodiment the cell population comprising pancreatic cells is a Slc30a8/ZnT-8-positive fraction.

In one embodiment the culture of pancreatic endocrine cells obtained by the method described herein is further separated in a beta cell-positive fraction. In one embodiment the culture of pancreatic endocrine cells obtained by the method described herein is further separated in a ptprn/IA2-positive fraction. In one embodiment the culture of pancreatic endocrine cells obtained by the method described herein is further separated in a Abcc8/Sur1-positive fraction. In one embodiment the culture of pancreatic endocrine cells obtained by the method described herein is further separated in a Slc30a8/ZnT-8-positive fraction. In one embodiment the cell population comprising pancreatic cells is a beta cell-positive fraction, including a Ptprn-positive fraction, an Abcc8-positive fraction, and/or a Slc30a8-positive fraction. Also, the culture of pancreatic endocrine cells obtained by any of the methods described herein may be further separated in a beta cell-positive/negative fraction, including a Ptprn-positive/negative fraction, an Abcb9-positive/negative fraction, and/or a Slc30a8-positive/negative fraction.

A person skilled in the art will realise that all details herein or in the section above relating to methods of separating, although explicitly stated for G6PC2-encoded beta cell surface tag, may also be applied to other extracellular proteins. Such extracellular protein include proteins selected from the group consisting of DNER, DDR1, prominin 1 (also known as CD133), and CD49f. Specifically, such extracellular protein may be DNER or DDR1.

During differentiation of embryonic stem (ES) cells into beta cells it is expected that other cells types, such as, e.g., neural cells, and non-pancreatic endoderm will also be produced. ES cells can be differentiated to endodermal cells by activin A and then to pancreatic cells. Once the pancreatic fate has been acquired the wanted cells can be isolated. In one embodiment a reagent binding G6PC2-encoded beta cell surface tag will specifically bind to the fully differentiated beta cells of the pancreas. In one embodiment using a G6PC2-encoded beta cell surface tag as a marker will provide a cell population comprising fully differentiated beta cells with a low ratio of other cell types, such as less than 20%, less than 15%, less than 10%, less than 5%, less than 2%, or no other cell types, such as endodermal cells other than pancreatic cells. In one embodiment a double sorting is carried out using a reagent binding G6PC2-encoded beta cell surface tag and one or more additional binding reagents. In one embodiment compositions comprising pancreatic endocrine cells substantially free of other cell types may be produced. In one embodiment compositions comprising fully differentiated beta cells substantially free of other cell types may be produced. In one embodiment the expression “substantially free of” is for cell cultures or cell populations to be understood as a cell culture or cell population comprising less than 20% other cell types than fully differentiated beta cells in relation to the total number of cells.

In one embodiment the invention relates to a method wherein the resulting cell population consists of at least 30%, such as at least 40%, at least 50%, at least 60%, at least 70%, at least 80%, at least 90%, or at least 95% fully differentiated beta cells.

In one embodiment the invention relates to a method wherein the starting cell population is of endodermal origin and the resulting cell population consists of at least 30%, such as at least 40%, at least 50%, at least 60%, at least 70%, at least 80%, at least 90%, or at least 95% fully differentiated beta cells.

In one embodiment the additional binding reagent is selected from the group consisting of Kir6.2 and Sur1. In one embodiment the additional binding reagent is selected from the group consisting of DNER, DDR1, prominin 1 (also known as CD133), and CD49f binding reagent. In embodiment the invention the additional binding reagent is DNER or Ddr1 binding reagent.

In one embodiment the invention relates to an isolated cell selected from the group consisting of fully differentiated beta cells obtained by any of the methods defined herein.

In one embodiment the invention relates to a composition comprising isolated fully differentiated beta cells obtained by a method as defined herein.

### Cellular Differentiation Markers

There are a number of cellular markers that can be used to identify populations of pancreatic cells. Donor cells isolated and cultured begin to display various phenotypic and genotypic indicia of differentiated pancreatic cells. Examples of the phenotypic and genotypic indicia include various molecular markers present in the facultative progenitor cell population that are modulated (e.g., either up or down regulated). These molecular markers include CK-19 or the Pdx1/Nkx6.1/Ptf1a triple positive cell, which is hypothesized to be a marker of the pancreatic facultative stem cell (i.e. the multi-potent pancreatic stem cell).

Typically, mammalian stem cells proceed through a number of developmental stages as they mature to their ultimate developmental endpoint. Developmental stages often can be determined by identifying markers present or absent in developing cells. Because human endocrine cells develop in a similar manner, various markers can be used to identify cells as they transition from a stem cell-like phenotype to pseudo-islet phenotype.

The expression of markers in cells induced to proliferate or differentiate by the methods of the present invention bears some similarity to the sequence of marker expression in normal human pancreas development. Very early in development, the primordial epithelial cells express Pdx-1, an early cellular marker that is a homeodomain nuclear factor. As the cells develop, they begin to bud out and form a duct. These cells express cytokeratin 19, a marker for epithelial ductal cells, and temporally express Ngn-3 leading developmentally to endocrine cells. As these cells continue to develop towards endocrine cells, they gain the ability to express insulin, somatostatin, glucagon, ghrelin, or pancreatic polypeptide. The final differentiated cells are only able to express one and become the alpha cells (glucagon), beta cells (insulin), delta cells (somatostatin), epsilon cells (ghrelin), and PP-cells.

Other proteins, such as ptprn/IA2, Abcc8/Sur1, and Slc30a8/ZnT-8, can be used to identify, enrich, and/or isolate fully differentiated beta cells.

Markers of interest are molecules that are expressed in temporal- and tissue-specific patterns in the pancreas (see Hollingsworth, Ann N Y Acad Sci 880: 38-49 (1999)). These molecular markers are divided into three general categories: transcription factors, notch pathway markers, and intermediate filament markers. Examples of transcription factor markers include Pdx-1, NeuroD, Nkx-6.1, Isl-1, Pax-6, Pax-4, Ngn-3, and HES-1.

Examples of notch pathway markers include Notch1, Notch2, Notch3, Notch4, Jagged1, Jagged2, Dill, and RBPjk. Examples of intermediate filament markers include CK19 and nestin. Examples of markers of precursors of pancreatic beta cells include Pdx-1, Pax-4, Ngn-3, and Hb9. Examples of markers of fully differentiated pancreatic beta cells include insulin, ptprn/IA2, Abcc8/Sur1, and Slc30a8/ZnT-8.

Insulin mRNA Expression

One marker that may be used to characterize pancreatic cell identity, differentiation, or maturity is the level of insulin mRNA. For example, the intermediate cell population of the present invention show expression of insulin mRNA within a defined range. Method for quantitating insulin mRNA include Northern blots, nuclease protection, and primer extension.

In one embodiment RNA is extracted from a population of cultured cells, and the amount of proinsulin message is measured by quantitative reverse transcription PCR. Following reverse transcription, insulin cDNA is specifically and quantitatively amplified from the sample using primers hybridizing to the insulin cDNA sequence, and amplification conditions under which the amount of amplified product is related to the amount of mRNA present in the sample (see, e.g., Zhou et al., J Biol Chem 272: 25648-51 (1997)). Kinetic quantification procedures are preferred due to the accuracy with which starting mRNA levels can be determined.

Frequently, the amount of insulin mRNA is normalized to a constitutively expressed mRNA, such as actin, which is specifically amplified from the same RNA sample using actinspecific primers. Thus, the level of expression of insulin mRNA may be reported as the ratio of insulin mRNA amplification products to actin mRNA amplification products, or simply the insulin: actin mRNA ratio. The expression of mRNAs encoding other pancreatic hormones (e.g., somatostatin or glucagon) may be quantified by the same method. Insulin and actin mRNA levels can also be determined by in situ hybridization and then used to determine insulin: actin mRNA ratios. In situ hybridization methods are known to those skilled in the art.

### Methods of Expansion

In one embodiment the invention relates to a method of expanding the numbers of fully differentiated beta cells, the method comprising: obtaining cells purified according to the method described above or herein and then subsequently culturing the obtained cells under conditions which facilitate expansion of the fully differentiated beta cells.

A protocol for expansion of pancreatic cells derived from fetal/adult tissue and stem cells is exemplified by, but not limited to, the protocols described in Heimberg, H. et al. (2000), Diabetes 49, 571-9; Heremans, Y. et al. (2002), J Cell Biol 159, 303-12; Miralles, F. et al. (1998), Development 125, 1017-24; and Miralles, F. et al. (1999), Dev Dyn 214, 116-26.

### Functional Assays

One of the important functions of a beta cell is to adjust its insulin secretion according to the glucose level. Typically, a static glucose stimulation (SGS) assay can be performed on the proliferating adherent pancreatic cells to identify whether they are able to secrete insulin in response to different glucose levels. Cells are generally cultured on an appropriate substrate until nearly confluent. Three days prior to the SGS test, the culture medium is replaced by a medium of similar character but lacking insulin and containing only 1 g/L of glucose. The medium is changed each day for three days and the SGS test is performed on day four.

Before the test, the culture medium may be collected for glucose and insulin analysis. To prepare cells for the test, cells are washed twice with Dulbecco's phosphate-buffered saline (DPBS)+0.5% BSA, incubating for 5 minutes with each wash, and then once with DPBS alone, also incubating for 5 minutes. After washing, the cells are incubated with 10 ml (in a 100 mm dish) or 5 ml (in a 60 mm dish) of Krebs-Ringers SGS solution with 60 mg/dl glucose (KRB-60) for 30 minutes in a 37° C. incubator. This incubation is then repeated.

To perform the SGS assays, cells are incubated in 3 ml (100 mm dish) or 4 ml (T75 flask) or 2 ml (60 mm dish) KRB-60, at 37° C. for 20 minutes. The medium is aspirated and spun, and is collected for insulin assay as LG-1 (low glucose stimulated step). KRB-450+theo (KRB with 450 mg/dl glucose and 10 mM theophylline) is then added with the same volume as above, and cells are cultured under the same condition as above. The supernatant is collected for insulin assay as HG (high glucose stimulated). The cells are then incubated again with KRB-60 and the medium collected as LG-2, and another time as LG-3. The media are collected for insulin analysis, and stored at −20° C. until insulin content is determined by radioimmunoassay (RIA) or other suitable assay.

The results of the SGS test are often expressed as a stimulation index, defined as the HG insulin value divided by the LG-1 insulin value. Generally, a stimulation index of about 2 or greater is considered to be a positive result in the SGS assay, although other values (e.g., 1.5, 2.5, 3.0, 3.5, etc.) may be used to define particular cell populations.

### Treatment Methods

In one embodiment the invention relates to a method of providing pancreatic endocrine function to a mammal deficient in its production of at least one pancreatic hormone wherein cells are obtained by any of the methods described above or herein, the method further comprising the steps of: implanting into the mammal the obtained cells in an amount sufficient to produce a measurable amount of at least one pancreatic hormone in the mammal.

Those skilled in the art will recognize that cells selected using G6PC2-encoded beta cell surface tag or further cultured cells provide a renewable resource for implantation and restoration of pancreatic function in a mammal.

If desired by the user, cells positive for G6PC2-encoded beta cell surface tag can be encapsulated before implantation.

Differentiation of pancreatic cells before implantation into the mammal may be to the stages of differentiation selected from the group consisting of fully differentiated endocrine cells and fully differentiated beta cells, including glucose responsive insulin producing beta cells, i.e. equivalent to beta cells of the isolated islet of Langerhans. An example of implantation isolated islet of Langerhans using the so-called Edmonton protocol can be found in Shapiro A M (2000), N Engl J. Med.; 343(4):230-8.

Encapsulation of the cells positive for G6PC2-encoded beta cell surface tag results in the formation of cellular aggregates in the capsules. Encapsulation can allow the pancreatic cells to be transplanted into a type 1 diabetic host, while minimizing the immune response of the host animal. The porosity of the encapsulation membrane can be selected to allow secretion of biomaterials, like insulin, from the capsule, while limiting access of the host's immune system to the foreign cells.

Encapsulation methods are known in the art and are disclosed in the following references: van Schelfgaarde & de Vos, J. Mol. Med. 77: 199-205 (1999), Uludag et al. Adv. DrugDel Rev. 42: 29-64 (2000) and U.S. Pat. Nos. 5,762,959, 5,550,178, and 5,578,314.

Encapsulation methods are described in detail in application PCT/US02/41616; herein incorporated by reference.

Implantation or transplantation into a mammal and subsequent monitoring of endocrine function may be carried out according to methods commonly employed for islet transplantation; see, e.g., Ryan et al., Diabetes 50: 710-19 (2001); Peck et al., Ann Med 33: 186-92 (2001); Shapiro et al., N Engl J Med 343 (4): 230-8 (2000); Carlsson et al., Ups J Med Sci 105 (2): 107-23 (2000) and Kuhtreiber, WM, Cell Encapsulation Technology and Therapeutics, Birkhauser, Boston, 1999. Preferred sites of implantation include the peritoneal cavity, the liver, and the kidney capsule.

A person skilled in the art will realise that in the case of carrying out transplantation using pancreatic cells comprising immature pancreatic cells, measurement of beta cell function, such as measurement of pancreatic hormone and blood glucose levels, should be carried out at least 2 weeks, such as at least 4 weeks, at least 6 weeks, or at least 8 weeks after the transplantation. In contrast, when carrying out transplantation using differentiated pancreatic cells beta cell function, such as measurement of pancreatic hormone and blood glucose levels, should be carried out right after transplantation, such as before 12 hours, before 24 hours, or before 36 hours after the transplantation.

A person skilled in the art will be able to determine an appropriate dosage of the number of fully differentiated beta cells or microcapsules for an intended recipient. The dosage will depend on the insulin requirements of the recipient. Insulin levels secreted by the defined number of beta cells or microcapsules can be determined immunologically or by amount of biological activity. The recipient's body weight can also be taken into account when determining the dosage. If necessary, more than one implantation can be performed as the recipient's response to the (optionally encapsulated) cells is monitored. Thus, the response to implantation can be used as a guide for the dosage of (optionally encapsulated) cells. (Ryan et al., Diabetes 50: 710-19 (2001))

The function of (optionally encapsulated) cells in a recipient can be determined by monitoring the response of the recipient to glucose. Implantation of the (optionally encapsulated) cells can result in control of blood glucose levels. In addition, evidence of increased levels of pancreatic endocrine hormones, insulin, C-peptide, glucagon, and somatostatin can indicate function of the transplanted (optionally encapsulated) cells.

One skilled in the art will recognize that control of blood glucose can be monitored in different ways. For example, blood glucose can be measured directly, as can body weight and insulin requirements. Oral glucose tolerance tests can also be given. Renal function can also be determined as can other metabolic parameters. (Soon-Shiong, P. et al., PNAS USA 90: 5843-5847 (1993); Soon-Shiong, P. et al., La71cet 343: 950-951 (1994)).

The term “insulin producing endocrine pancreatic cells” as used herein refers to cells that produce insulin and secrete insulin in a blood glucose dependent manner.

In one embodiment the cell population comprising pancreatic cells are isolated (this invention) from a cultured source. The isolated cells are then used for example in further culturing or for microencapsultation according to the microencapsulation method of U.S. Pat. No. 5,762,959.

The term “providing pancreatic function to a mammal in need of such function” refers to a method of producing pancreatic hormones within the body of a mammal unable to produce such hormones on its own. In one embodiment the pancreatic hormone is selected from the group consisting of insulin, glucagon, somatostatin, pancreatic polypeptide, and ghrelin. In one embodiment insulin is produced in the body of a diabetic mammal. The pancreatic function is provided by implanting or transplanting insulin producing pancreatic cells, produced by the methods of this disclosure into the mammal. The number of aggregates implanted is an amount sufficient to produce a measurable amount of insulin in the mammal. The insulin can be measured by Elisa assay or Radioimmunoassay or by other detection methods known to those skilled in the art, including assays for insulin function, such as maintenance of blood glucose levels.

Insulin is co-secreted with C-peptide in equimolar amounts. Thus, insulin secretion activity can also be measured by detecting C-peptide in the blood. In one embodiment the provision of pancreatic function is sufficient to decrease or eliminate the dependence of the mammal on insulin produced outside the body.

“Encapsulation” refers to a process where cells are surrounded by a biocompatible acellular material, such as sodium alginate and polylysine. Preferably small molecules, like sugars and low molecular weight proteins, can be taken up from or secreted into an environment surrounding the encapsulated cells. At the same time access to the encapsulated cells by larger molecules and immune cells is limited.

“Implanting” is the grafting or placement of the cells into a recipient. It includes encapsulated cells and non-encapsulated. The cells can be placed subcutaneously, intramuscularly, intraportally or interperitoneally by methods known in the art.

In one embodiment the step of separating is done by fluorescence activated cell sorting. In one embodiment the step of separating is done by panning. In one embodiment the step of separating is done by fluidised bed.

In one embodiment the invention relates to the use of a reagent binding G6PC2-encoded beta cell surface tag to identify or select cells that express G6PC2-encoded beta cell surface tag as a cell surface marker. In one embodiment the invention relates to the simultaneous or sequential use of a reagent binding G6PC2-encoded beta cell surface tag to identify or select cells that express G6PC2-encoded beta cell surface tag as a cell surface marker in combination with one or more additional binding reagents, which are subjected to the same step(s) of analysis as the reagent binding G6PC2-encoded beta cell surface tag. In one embodiment the additional binding reagent is selected from the group consisting of Kir6.2 and Sur1. In one embodiment the additional binding reagent is selected from the group consisting of DNER, DDR1, prominin 1 (also known as CD133), and CD49f binding reagent. In one embodiment the additional binding reagent is DNER or DDR1 binding reagent.

In one embodiment of the invention the additional binding reagents are selected from the group consisting of Ptprn/IA2, Abcc8/Sur1, and Slc30a8/ZnT-8 binding reagent, which can be used to isolate early and fully differentiated endocrine cells.

In one embodiment the invention relates to the method of treating type I diabetes by providing pancreatic function to a mammal in need of such function.

### Embodiments of the Invention

1. An isolated peptide encoded by G6PC2, wherein said peptide comprises i) an extracellular part and ii) a deletion in one or more exons of G6PC2, provided that said peptide is not MDFLHRNGVLIIQHLQKDYRAYYTFLNFMSNVGDPRNIFFIYFPLCFQFNQTVGTKMIWVAVIGDWLNLIFKWKSIWPCNGRILCLVCHGNRCPEPHCLWDG.
2. An isolated peptide according to any of the preceding embodiments, wherein said peptide consists of an extracellular part.
3. An isolated peptide according to any of the preceding embodiments, which comprises a sequence contained in one or more exons selected from the group consisting of exon 1, exon 2, exon 3, exon 4 and exon 5 of G6PC2.
4. An isolated peptide according to any of the preceding embodiments, which comprises a deletion in a sequence selected from one or more from the group consisting of exon 1, exon 2, exon 3, exon 4 and exon 5 of G6PC2.
5. An isolated peptide according to any of the preceding embodiments, wherein said peptide comprises a sequence selected from the group consisting of MDFLHRNGVLIIQHLQK-DYRAYYT (SEQ ID NO: 3), MLVAEAFEHTPGIQTASLGT (SEQ ID NO: 4), LLSCRGGNNY (SEQ ID NO: 5), HMLMKQSGKKSQ (SEQ ID NO: 6), MLVAEAFEHTPGIQ (SEQ ID NO: 8) as well as variants thereof.
6. An isolated peptide according to any of the preceding embodiments, wherein said peptide consists of a sequence selected from the group consisting of MDFLHRNGVLIIQHLQK-DYRAYYT (SEQ ID NO: 3), MLVAEAFEHTPGIQTASLGT (SEQ ID NO: 4), LLSCRGGNNY (SEQ ID NO: 5), HMLMKQSGKKSQ (SEQ ID NO: 6), MLVAEAFEHTPGIQ (SEQ ID NO: 8) as well as variants thereof.
7. An isolated peptide according to any of the preceding embodiments, wherein said peptide comprises a naturally occurring amino acid sequence of at least 80%, such as at least 90%, or at least 96% identity to an amino acid sequence selected from the group consisting of MDFLHRNGVLIIQHLQKDYRAYYT (SEQ ID NO: 3), MLVAEAFEHTPGIQTASLGT (SEQ ID NO: 4), LLSCRGGNNY (SEQ ID NO: 5), HMLMKQSGKKSQ (SEQ ID NO: 6), MLVAEAFEHTPGIQ (SEQ ID NO: 8).
8. An isolated peptide according to any of the preceding embodiments, wherein said peptide is a fragment of the isolated peptide as defined in embodiments 1-7 or a part of the full-length IGRP.9. An isolated nucleotide encoding a peptide as defined in any of embodiments 1-8.
10. An isolated cell expressing a peptide as defined in any of embodiments 1-8.
11. A method of identification of fully differentiated beta cells, the method comprising contacting a cell population comprising pancreatic cells with a reagent binding a G6PC2-encoded beta cell surface tag, wherein the G6PC2-encoded beta cell surface tag optionally is as defined in any of embodiments 1-8.
12. A method of obtaining a culture of fully differentiated beta cells, the method comprising: contacting a cell population comprising pancreatic cells with a reagent binding a G6PC2-encoded beta cell surface tag and separating the cells that binds the reagent binding a G6PC2-encoded beta cell surface tag in a fraction of cells positive for a G6PC2-encoded beta cell surface tag from cells that do not bind the reagent binding a G6PC2-encoded beta cell surface tag, wherein the G6PC2-encoded beta cell surface tag optionally is as defined in any of embodiments 1-87.
13. A method of providing pancreatic endocrine function to a mammal deficient in its production of at least one pancreatic hormone wherein cells are obtained by the method according to any one of embodiments 11-12, the method further comprising the steps of: implanting into the mammal the obtained cells in an amount sufficient to produce a measurable amount of at least one pancreatic hormone in the mammal.
14. The method according to any one of embodiments 11-13, wherein said at least one pancreatic hormone is selected from the group consisting of insulin, glucagon, somatostatin, pancreatic polypeptide, and ghrelin.
15. A method of quantifying cells positive for a G6PC2-encoded beta cell surface tag comprising pancreatic cells by a) contacting the cells with a reagent binding a G6PC2-encoded beta cell surface tag; and b) determining the quantity of cells that exhibit a G6PC2-encoded beta cell surface tag as a cell surface marker (cells positive for G6PC2-encoded beta cell surface tag), wherein the G6PC2-encoded beta cell surface tag optionally is as defined in any of embodiments 1-8.
16. A method for the optimisation of an in vitro protocol, wherein the number of cells expressing a G6PC2-encoded beta cell surface tag (cells positive for a G6PC2-encoded beta cell surface tag) is periodically monitored, wherein the G6PC2-encoded beta cell surface tag optionally is as defined in any of embodiments 1-8.
17. The method according to any one of embodiments 11-16, wherein one or more additional binding reagents are used either simultaneously or sequentially in combination with the reagent binding a G6PC2-encoded beta cell surface tag.
18. The method according to embodiment 17, wherein an additional binding reagent is selected from the group consisting of DNER, DDR1, prominin 1 (also known as CD133), and CD49f binding reagents.
19. The method according to embodiment 17, wherein the additional binding reagent is a DNER or DDR1 binding reagent.
20. The method according to any one of the embodiments 11-19, wherein the cell population comprising pancreatic cells is obtained from a pancreas.
21. The method according to any one of the embodiments 11-19, wherein the cell population comprising pancreatic cells is obtained from a somatic cell population.
22. The method according to any one of the embodiments 11-19, wherein the cell population comprising pancreatic cells is obtained from pluripotent cells, such as ES cells.
23. The method according to any of embodiments 11-22, wherein the cell population comprising pancreatic cells is of mammalian origin.
24. The method according to any of embodiments 11-23, wherein the cell population comprising pancreatic cells is of human origin.
25. The method according to any one of the embodiments 11-24, wherein the cell population comprising pancreatic cells is a beta cell-positive fraction.
26. The method according to any one of the embodiments 11-24, wherein the cell population comprising pancreatic cells is a ptprn/IA2-positive fraction.
27. The method according to any one of the embodiments 11-24, wherein the cell population comprising pancreatic cells is an Abcc8/Sur1-positive fraction.
28. The method according to any one of the embodiments 11-24, wherein the cell population comprising pancreatic cells is a Slc30a8/ZnT-8-positive fraction.
29. The method according to any one of the embodiments 11-24, wherein the culture of pancreatic endocrine cells obtained by the method according to any of the preceding embodiments is further separated in a beta cell-positive fraction.
30. The method according to any one of the embodiments 11-24, wherein the culture of pancreatic endocrine cells obtained by the method according to any of the preceding embodiments is further separated in a ptprn/IA2-positive fraction.
31. The method according to any one of the embodiments 11-24, wherein the culture of pancreatic endocrine cells obtained by the method according to any of the preceding embodiments is further separated in an Abcc8/Sur1-positive fraction.
32. The method according to any one of the embodiments 11-24, wherein the culture of pancreatic endocrine cells obtained by the method according to any of the preceding embodiments is further separated in a Slc30a8/ZnT-8-positive fraction.
33. The method according to any of embodiments 11-32, wherein the reagent binding a G6PC2-encoded beta cell surface tag is an antibody that specifically binds to a G6PC2-encoded beta cell surface tag.
34. The method according to any of embodiments 11-33, wherein the step of separating or monitoring is done by fluorescence activated cell sorting.
35. The method according to any of embodiments 11-34, wherein the step of separating is done by panning.
36. The method according to embodiment 13, wherein said at least one pancreatic hormone is insulin.
37. The method according to embodiment 11-37, wherein the mammal is a human being.
38. The method according to any of embodiments 11-37, wherein the cells are fully differentiated beta cells.
39. The method according to any of embodiments 11-38, wherein the G6PC2-encoded beta cell surface tag is as defined in any of the embodiments 1-8.
40. The method according to any of embodiments 11-38, wherein the G6PC2-encoded beta cell surface tag is a part of the full-length IGRP.
41. An isolated fully differentiated endocrine beta cell obtained by a method as defined in any of the embodiments 11-40.
42. A composition comprising isolated fully differentiated beta cells obtained by a method as defined in any of the embodiments 11-40.
43. Use of a reagent binding a G6PC2-encoded beta cell surface tag to identify or select cells that express a G6PC2-encoded beta cell surface tag as a cell surface marker, wherein the G6PC2-encoded beta cell surface tag optionally is as defined in any of embodiments 1-8.
44. Use of a G6PC2-encoded beta cell surface tag as a cell surface marker to obtain a culture of pancreatic endocrine cells, wherein the G6PC2-encoded beta cell surface tag optionally is as defined in any of embodiments 1-8.
45. Use according to embodiment 44, wherein one or more further cell surface markers are used simultaneously or sequentially to obtain a culture of pancreatic endocrine cells.
46. Use according to embodiment 45, wherein a further cell surface marker is selected from the group consisting of DNER protein, DDR1 protein, prominin 1 (also known as CD133), and CD49f.
47. Use according to embodiment 45, wherein a further cell surface marker is DNER protein or DDR1 protein.
48. Use according to any of embodiments 43-47, wherein the G6PC2-encoded beta cell surface tag is as defined in any of the embodiments 1-8.
49. Use according to any of embodiments 43-48, wherein the G6PC2-encoded beta cell surface tag is a part of the full-length IGRP.
50. An antibody that specifically binds to a G6PC2-encoded beta cell surface tag, wherein the G6PC2-encoded beta cell surface tag optionally is as defined in any of embodiments 1-8.
51. An antibody according to embodiment 50, wherein the G6PC2-encoded beta cell surface tag is as defined in any of the embodiments 1-8.
52. An antibody according to embodiment 50, wherein the G6PC2-encoded beta cell surface tag is a part of the full-length IGRP.
53. An isolated peptide according to any of the preceding embodiment, wherein said peptide is present and/or detectable on the outer surface of a cell.

### EXAMPLES

### Example 1

### Co-Localisation of Insulin Expression and G6PC2

Adult human pancreas tissue was harvested and fixed over night (0/N) in 4% paraformaldehyde (PFA) in PBS. Tissue was equilibrated in 30% sucrose in PBS and embedded in TissueTech. 8 μm sections were cut and stored at −80° C. Sections were thawed at room temperature (RT), washed in PBS, microwaved in 0.01 M citrate buffer and incubated with 1% H₂O₂ in PBS for 30 minutes and subsequently blocked with 0.5% TNB blocking reagent (from Perkin-Elmer). Anti-G6PC2 was raised against MDFLHRNGVLIIQHLQKDYRAYYTFC coupled to KLH (keyhole limpet hemocyanin) via the C-terminal cysteine in rabbits. Rabbit anti-G6PC2 (Rb-anti-G6PC2) 1:9000, guinea pig anti-insulin (Gp anti-Ins) 1:150 were then added and incubated O/N at RT. The following day sections were washed in PBS and biotinylated donkey anti-rabbit and donkey anti-guinea pig-cy2 antibody were added and incubated for 45 minutes. After washing the cells in PBS streptavidin-HRP was added and incubated for 15 minutes. Subsequently, the cells were washed in PBS. Finally, tyramid-cy3 was added to develop the staining.

The results showed robust expression of G6PC2 in the Islet of Langerhans. Weaker expression was also observed outside the Islet of Langerhans. In the Islet of Langerhans the G6PC2 signal co-localised with insulin, demonstrating that G6PC2 is expressed in the betacell.

### Example 2

### In Vitro Protocol Optimisation

In one embodiment an in vitro culture comprising pancreatic cells is periodically monitored for expression of G6PC2-encoded beta cell surface tag. In one embodiment one or more additional markers may be used in combination with G6PC2-encoded beta cell surface tag either simultaneously or sequentially. In one embodiment the additional marker is selected from the group consisting of DNER, prominin 1 (also known as CD133), CD49f, DISP2, LRP11, SLC30A8 and SEZ6L2. In one embodiment the additional marker is selected from the group consisting of DNER, DDR1 protein, prominin 1 (also known as CD133), and CD49f. In one embodiment the additional marker is DNER or DDR1 protein.

For efficient optimization of differentiation of embryonic stem cells it is very important to have markers identifying the various stages of development of pancreatic cells towards fully differentiated endocrine cells and/or fully differentiated beta cells. G6PC2-encoded surface tag may be used to pinpoint important and specific stages of cellular differentiation which until now not have been possible to detect using other markers. G6PC2-encoded surface tag may be used in combination with one or more additional markers.

Cells expressing G6PC2-encoded beta cell surface tag can be identified, enriched, and/or isolated using methods as described herein, e.g., FACS, panning, MACS.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1-15. (canceled)

16. A method of identification of fully differentiated beta cells, the method comprising contacting a cell population comprising pancreatic cells with a reagent binding a G6PC2-encoded beta cell surface tag.

17. A method of providing pancreatic endocrine function to a mammal deficient in its production of at least one pancreatic hormone wherein cells are obtained by the method of claim 16, the method further comprising the step of implanting into the mammal the obtained cells in an amount sufficient to produce a measurable amount of at least one pancreatic hormone in the mammal.

18. The method according to claim 17, wherein said at least one pancreatic hormone is selected from the group consisting of insulin, glucagon, somatostatin, pancreatic polypeptide, and ghrelin.

19. The method according to claim 16, wherein one or more additional binding reagents are used either simultaneously or sequentially in combination with the reagent binding a G6PC2-encoded beta cell surface tag.

20. The method according to claim 19, wherein an additional binding reagent is selected from the group consisting of DNER, DDR1, prominin 1 (also known as CD133), and CD49f binding reagents.

21. The method according to claim 16, wherein the cells are fully differentiated beta cells.

22. The method according to claim 16, wherein the G6PC2-encoded beta cell surface tag is a part of the full-length IGRP.

23. A method of obtaining a culture of fully differentiated beta cells, the method comprising: contacting a cell population comprising pancreatic cells with a reagent binding a G6PC2-encoded beta cell surface tag; and separating the cells that binds the reagent binding a G6PC2-encoded beta cell surface tag in a fraction of cells positive for a G6PC2-encoded beta cell surface tag from cells that do not bind the reagent binding a G6PC2-encoded beta cell surface tag.

24. A method of providing pancreatic endocrine function to a mammal deficient in its production of at least one pancreatic hormone wherein cells are obtained by the method of claim 23, the method further comprising the steps of: implanting into the mammal the obtained cells in an amount sufficient to produce a measurable amount of at least one pancreatic hormone in the mammal.

25. The method according to claim 24, wherein said at least one pancreatic hormone is selected from the group consisting of insulin, glucagon, somatostatin, pancreatic polypeptide, and ghrelin.

26. The method according to claim 23, wherein one or more additional binding reagents are used either simultaneously or sequentially in combination with the reagent binding a G6PC2-encoded beta cell surface tag.

27. The method according to claim 26, wherein an additional binding reagent is selected from the group consisting of DNER, DDR1, prominin 1 (also known as CD133), and CD49f binding reagents.

28. The method according to claim 23, wherein the cells are fully differentiated beta cells.

29. The method according to claim 23, wherein the G6PC2-encoded beta cell surface tag is a part of the full-length IGRP.

30. A method of quantifying cells positive for a G6PC2-encoded beta cell surface tag comprising by a) contacting the cells with a reagent binding a G6PC2-encoded beta cell surface tag; and b) determining the quantity of cells that exhibit a G6PC2-encoded beta cell surface tag as a cell surface marker.

31. The method of quantifying cells positive for a G6PC2-encoded beta cell surface tag of claim 30, wherein the quantity of cells expressing a G6PC2-encoded beta cell surface tag is periodically monitored.

32. The method according to claim 30, wherein one or more additional binding reagents are used either simultaneously or sequentially in combination with the reagent binding a G6PC2-encoded beta cell surface tag.

33. The method according to claim 32, wherein an additional binding reagent is selected from the group consisting of DNER, DDR1, prominin 1 (also known as CD133), and CD49f binding reagents.

34. The method according to claim 30, wherein the cells are fully differentiated beta cells.

35. The method according to claim 30, wherein the G6PC2-encoded beta cell surface tag is a part of the full-length IGRP.

36. An isolated fully differentiated endocrine beta cell obtained by a method as defined in claim 16.

37. A composition comprising isolated fully differentiated beta cells obtained by a method as defined in claim 16.

38. Use of a reagent binding a G6PC2-encoded beta cell surface tag to identify or select cells that express a G6PC2-encoded beta cell surface tag as a cell surface marker.

39. Use of a G6PC2-encoded beta cell surface tag as a cell surface marker to obtain a culture of pancreatic endocrine cells.

40. An antibody that specifically binds to a G6PC2-encoded beta cell surface tag.
